(19) United States
(12) Patent Application Publication
(10) Pub. No.: US 2026/0182976 A1
(43) Pub. Date: Jul. 2, 2026

(54) LAPAROSCOPIC DIFFICULT SPECIMEN RETRIEVAL BAG

(71) Applicant: The First Affiliated Hospital of Wenzhou Medical University, Wenzhou (CN)

(72) Inventors: Xiaodong ZHANG, Wenzhou (CN); Baiwang ZHU, Wenzhou (CN); Zhijie YU, Wenzhou (CN); Chenxiao ZHENG, Wenzhou (CN); Yifei PAN, Wenzhou (CN)

(73) Assignee: The First Affiliated Hospital of Wenzhou Medical University, Wenzhou, ZJ (CN)

(21) Appl. No.: 19/428,353

(22) Filed: Dec. 22, 2025

(30) Foreign Application Priority Data
Dec. 31, 2024 (CN) 202411986574.0

(51) Int. Cl. A61B 17/00; A61M 25/10

(52) U.S. Cl. CPC A61B 17/00234; A61B 2017/00287; A61B 2017/00867; A61M 25/1018

(57) ABSTRACT

Disclosed is a laparoscopic difficult specimen retrieval bag, including a head portion with an opening on one side, a body portion, and a neck portion connecting the head portion and the body portion, where the body portion includes an inner cavity and an outer cavity, the inner cavity is in communication with the neck portion, the outer cavity is provided with an air inlet hole and an air outlet hole, the air inlet hole is used for being in communication with the inner cavity, and the air outlet hole is used for being in communication with the outside. During a specimen removal process, air is introduced into the inner cavity, so that a state that air pressure of the inner cavity of the retrieval bag is greater than air pressure of an abdominal cavity which is greater than external air pressure is always maintained in a surgical process.

### TECHNICAL FIELD

The disclosure relates to the technical field of retrieval bags, and in particular, to a laparoscopic difficult specimen retrieval bag.

### BACKGROUND

Laparoscopic appendectomy and cholecystectomy are common surgical procedures worldwide. In an abdominal cavity of a 13 mmHG pressure CO2 pneumoperitoneum created by an artificial pneumoperitoneum machine, a corresponding trocar is inserted through puncture holes made at different positions on an abdominal wall, and laparoscopic surgical instruments are placed inside the trocar to complete the predetermined surgical steps. Compared with traditional open laparotomy, they have advantages of minimal abdominal wall trauma, the ability to achieve no surgical incision on the abdominal wall, and rapid postoperative recovery for patients.

After the surgery, if the resected appendix or gallbladder specimen is of an appropriate volume, it can be directly removed from the trocar of the laparoscopic surgery or placed in a retrieval bag with a function of isolating contamination, and the specimen along with the retrieval bag can be directly removed together from the trocar puncture holes in the abdominal wall. Both of the above methods can prevent direct contact between the specimen and the tissue at the puncture holes of the human body, thus avoiding microbial contamination and subsequent infection. However, since the maximum puncture hole in conventional surgery is only 12 mm in size, a significant proportion of difficult specimens exist in clinical practice, such as difficult appendix specimens-due to swelling, stiffness, and flexion, and difficult gallbladder specimens-resulting from a large load of gallstones or large individual gallstone volume. These specimens cannot be quickly and relatively intactly removed from the trocar puncture holes in the abdominal wall.

According to the current clinical reality, surgeons face two options to achieve the goal of removing difficult specimens. Option 1: Enlarge the 12 mm trocar puncture hole in the abdominal wall to a length sufficiently accommodating the specimen. Disadvantages of this method: Firstly, for the surgery, due to the extension of the incision and the suturing of the extended incision after the operation, it will inevitably increase the surgical time. Secondly, for the patient, an excessively long surgical incision will remain postoperatively, and even incision-related complications such as incision infection, poor healing, and incisional hernia may occur. Option 2: Use sharp instruments such as forceps to perform “irregular and imprecise operations (including fragmented cutting, pulling, tearing, and trimming)” on the originally intact surgical specimen inside a traditional specimen bag that has been compressed and collapsed under the action of 13 mmHG pressure CO2 pneumoperitoneum, and thus the specimen is “fragmented”, the specimen volume is reduced, and then the fragmented specimens are removed in batches. Although this avoids incision extension, the specimen is fragmented into unrecognizable pieces. Disadvantages of this method: Firstly, due to the 13 mmHG high-pressure pneumoperitoneum in the abdominal cavity of the human body and the outward pulling action at the opening of the retrieval bag, various traditional retrieval bags will be flattened and collapsed, thus failing to provide sufficient necessary space for the surgeon to perform precise operations, inevitably prolonging the overall surgical time. Secondly, in clinical practice, some occult malignant tumors of the appendix and gallbladder exist, often complicated by acute or chronic inflammation. In specific cases, the fragmented specimens will pose unnecessary medical risks to the accuracy of postoperative pathological examinations. For instance, they will lead to an irreversible passive situation in pathological TNM staging, which is crucial for guiding the precise treatment of malignant tumors and evaluating survival prognosis.

### SUMMARY

To efficiently and intactly remove a difficult specimen from the body without additionally extending a surgical incision, this disclosure provides a laparoscopic difficult specimen retrieval bag.

The laparoscopic difficult specimen retrieval bag provided by this disclosure adopts the following technical solution:

The laparoscopic difficult specimen retrieval bag includes a head portion with an opening on one side, a body portion, and a neck portion connecting the head portion and the body portion, where the body portion includes an inner cavity and an outer cavity, the inner cavity is in communication with the neck portion, the outer cavity is provided with an air inlet hole and an air outlet hole, the air inlet hole is used for being in communication with the inner cavity, and the air outlet hole is used for being in communication with the outside.

By adopting the above technical solution, during use, after placing the retrieval bag into an abdominal cavity through a puncture cannula, the retrieval bag is expanded, and the specimen is placed into the inner cavity of the retrieval bag. Subsequently, the head portion and the neck portion of the retrieval bag are pulled out of the body surface from the trocar, the pneumoperitoneum is temporarily closed, and after the puncture cannula is removed, the trocar is immediately reinserted into the retrieval bag from the opening of the head portion of the retrieval bag, and the pneumoperitoneum is reopened. After gas enters the inner cavity containing the specimen through the puncture cannula, the air pressure inside the inner cavity is higher than the air pressure of the external abdominal cavity, so that the inner cavity is expanded to form a sufficiently large operating space, allowing an angle of the specimen to be accurately and quickly adjusted or the specimen to be efficiently and regularly segmented by a surgical instrument. Meanwhile, a part of the gas entering the inner cavity enters the outer cavity through the air inlet hole and is released into the abdominal cavity through the air outlet hole, so that the air pressure in the abdominal cavity is maintained in a state of being greater than the external air pressure, and a sufficient air cavity space is formed in the abdominal cavity. During this process, since the puncture hole in the abdominal wall is not completely sealed, the air in the abdominal cavity is also continuously released to the outside along with the puncture hole in the abdominal wall, so that a state that the air pressure of the inner cavity of the retrieval bag is greater than the air pressure of the abdominal cavity which is greater than the external air pressure is always maintained in a surgical process, and both the inner cavity and the abdominal cavity have sufficient space for surgeons to perform surgical operations.

Preferably, a memory metal ring for enabling the retrieval bag to automatically and elastically expand is disposed at the opening of the head portion.

By adopting the above technical solution, the retrieval bag can be automatically expanded in the abdominal cavity by using the memory metal ring, and at the same time, the retrieval bag can be compressed when passing through the puncture cannula without affecting the retrieval bag passing through the puncture cannula.

Preferably, a diaphragm is disposed in the inner cavity to divide the inner cavity into an upper cavity and a lower cavity, and a pore or a hole is disposed on the diaphragm.

By adopting the above technical solution, the separately disposed upper cavity and lower cavity allow specimen tissue to be retained in the upper cavity, and inherent contaminated fluids of the specimen, such as bile, pus, blood, and exudate of the gallbladder, and pus, blood, and exudate of the appendix, can enter the lower cavity through the diaphragm for collection.

Preferably, the air inlet hole is connected to a position in the inner cavity near a bottom of the upper cavity and above the diaphragm.

By adopting the above technical solution, the air inlet hole is disposed above the diaphragm, so that the gas needs to pass through a relatively large ascending space in the process of being discharged from the air outlet hole after entering the outer cavity through the air inlet hole, and the contaminated fluid is not easily brought out. Meanwhile, the air inlet hole is located above the diaphragm, so that the contaminated fluid is not easy to enter the outer cavity, thereby further reducing the possibility of the contaminated fluid being brought into the abdominal cavity to contaminate the abdominal cavity.

Preferably, the outer cavity is tubular and is located on one side of the inner cavity, isolation vanes are disposed inside the outer cavity at a position between the air inlet hole and the air outlet hole, and ventilation slits are disposed on the isolation vanes.

By adopting the above technical solution, the isolation vanes are disposed and the ventilation slits are disposed on the isolation vanes to achieve the effect of ventilation and fluid isolation, preventing the contaminated fluid from being brought into the abdominal cavity.

Preferably, at least two layers of the isolation vanes are disposed, and the ventilation slits on adjacent isolation vanes are disposed in a staggered manner.

By adopting the above technical solution, the at least two layers of the isolation vanes are disposed, so that during the process of the gas passing through an air inlet and being discharged from an air outlet, the gas needs to pass through a plurality of reciprocating airflow passages when passing through the ventilation slits of the isolation vanes. During this process, the gas is discharged smoothly, while the contaminated fluid is retained and is not discharged along with the airflow.

Preferably, the air inlet hole is connected to a position at a lower part in the inner cavity.

By adopting the above technical solution, a large amount of contaminated fluids in the inner cavity are not brought out to the outer cavity during the gas discharge process.

Preferably, the outer cavity is a rotational body cavity surrounding the inner cavity, and the air inlet hole and the air outlet hole are respectively located on opposite sides relative to the inner cavity.

By adopting the above technical solution, the airflow needs to bypass half of the outer circumference of the inner cavity when being discharged through the air inlet hole and the air outlet hole, so that the contaminated fluid is not easily brought out during the airflow discharge process.

Preferably, two layers of isolation vanes are disposed at a position above the air inlet hole inside the outer cavity, and a ventilation hole is disposed on each layer of the isolation vane.

By adopting the above technical solution, two layers of isolation vanes are disposed, so that when the airflow passes through the isolation vanes, the gas passes through the ventilation holes, and the fluid comes into contact with the isolation vanes during the airflow direction change, so that the contaminated fluid is retained in the outer cavity and is not easily discharged along with the airflow.

Preferably, the two layers of the isolation vanes are stacked together, the ventilation hole on the isolation vane at a lower layer is located on the same side as the air outlet hole relative to the inner cavity, and the ventilation hole on the isolation vane at an upper layer is located on the same side as the air inlet hole relative to the inner cavity.

By adopting the above technical solution, the two layers of the isolation vanes are disposed to be stacked together, so that when the air pressure below the isolation vanes is greater than the air pressure above the isolation vanes, the isolation vanes are slightly deformed, and a long and narrow airflow passage is formed between the two layers of the isolation vanes, so that the contaminated fluid cannot pass between the two layers of the isolation vanes without contacting the isolation vanes, and thus the contaminated fluid is not discharged from the outer cavity along with the airflow.

In conclusion, this disclosure has at least one beneficial technical effect as follows:

1. After gas enters the inner cavity containing the specimen through the puncture cannula, the air pressure inside the inner cavity is higher than the air pressure of the external abdominal cavity, so that the inner cavity is expanded to form a sufficiently large operating space, allowing an angle of the specimen to be accurately and quickly adjusted or the specimen to be efficiently and regularly segmented by a surgical instrument. Meanwhile, a part of gas entering the inner cavity enters the outer cavity through the air inlet hole and is released into the abdominal cavity through the air outlet hole, so that the air pressure in the abdominal cavity is maintained in a state of being greater than the external air pressure, and a sufficient air cavity space is formed in the abdominal cavity. During this process, since the puncture hole in the abdominal wall is not completely sealed, the air in the abdominal cavity is also continuously released to the outside along with the puncture hole in the abdominal wall, so that a state that the air pressure of the inner cavity of the retrieval bag is greater than the air pressure of the abdominal cavity which is greater than the external air pressure is always maintained in a surgical process, and both the inner cavity and the abdominal cavity have sufficient space for surgeons to perform surgical operations.

2. The at least two layers of the isolation vanes are disposed, so that during the process of the gas passing through an air inlet and being discharged from an air outlet, the gas needs to pass through a plurality of reciprocating airflow passages when passing through the ventilation slits of the isolation vanes. During this process, the gas is discharged smoothly, while the contaminated fluid is retained and not discharged along with the airflow.

### BRIEF DESCRIPTION OF THE DRAWINGS

FIG. 1 is a schematic structural diagram of Embodiment I in an expanded state;

FIG. 2 is an enlarged view of a position A in Embodiment I;

FIG. 3 is a schematic structural diagram of Embodiment II in an expanded state; and

FIG. 4 is a schematic structural diagram of Embodiment III in an expanded state.

Reference numerals in the accompanying drawings: 1. head portion; 2. neck portion; 3. body portion; 4. memory metal ring; 5. inner cavity; 6. outer cavity; 7. upper cavity; 8. lower cavity; 9. diaphragm; 10. air outlet hole; 11. air inlet hole; 12. isolation vane; 13. ventilation slit; 14. ventilation hole.

### DETAILED DESCRIPTION OF THE EMBODIMENTS

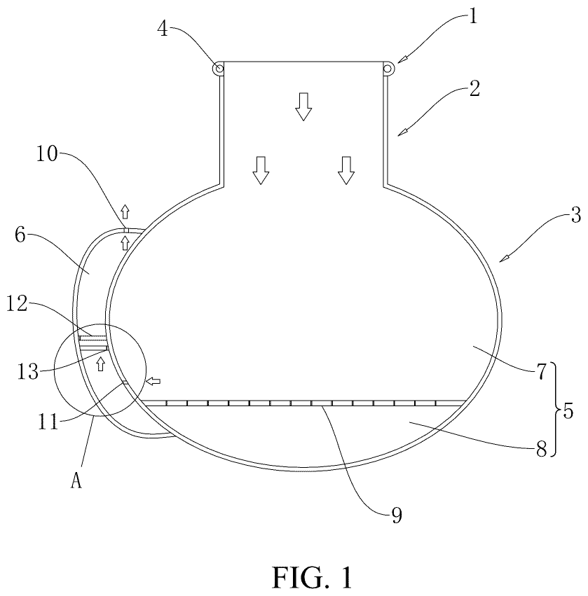

This disclosure is further detailed below with reference to FIGS. 1-4.

An embodiment of this disclosure provides a laparoscopic difficult specimen retrieval bag.

### Embodiment I

As shown in FIG. 1, the laparoscopic difficult specimen retrieval bag includes a head portion 1, a neck portion 2, and a body portion 3, where the neck portion 2 is used for connecting the head portion 1 and the body portion 3.

As shown in FIG. 1, a memory metal ring 4 is disposed in the opening of the head portion 1, and the memory metal ring is used for enabling the retrieval bag to be automatically and elastically expanded after being placed into the abdominal cavity through a puncture cannula, facilitating loading of the specimen.

As shown in FIG. 1, the body portion 3 includes an inner cavity 5 and an outer cavity 6. A diaphragm 9 is disposed in the inner cavity 5 to divide the inner cavity 5 into an upper cavity 7 and a lower cavity 8. A pore or a hole is disposed on the diaphragm 9, so that contaminated fluids carried by the specimen entering the inner cavity 5 (e.g., bile, pus, blood, and exudate of the gallbladder; pus, blood, and exudate of the appendix) flow into the lower cavity 8 under the action of gravity for collection.

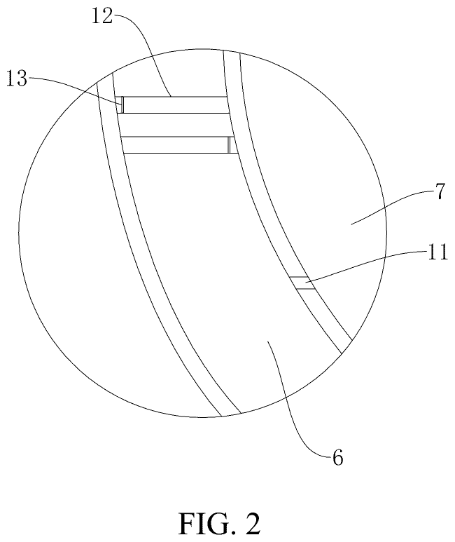

As shown in FIG. 1 and FIG. 2, the outer cavity 6 is connected to an outer side surface of the inner cavity 5, and the outer cavity 6 is tubular as a whole. An air outlet hole 10 is formed in an upper part of the outer cavity 6, and an air inlet hole 11 in communication with the inner cavity 5 is formed in a middle part of the outer cavity 6. The air inlet hole 11 is connected to a position in the inner cavity 5 near a bottom of the upper cavity 7 and above the diaphragm 9, and both the air inlet hole 11 and the air outlet hole 10 are small holes. At least two layers of isolation vanes 12 are disposed inside the outer cavity 6 at a position between the air inlet hole 11 and the air outlet hole 10. The isolation vanes 12 partition the entire outer cavity 6, and a ventilation slit 13 is formed on each layer of the isolation vane 12. The ventilation slits 13 on adjacent isolation vanes 12 are disposed in a staggered manner.

### Specific Use Process

During use, after placing the retrieval bag into an abdominal cavity through a puncture cannula of the trocar, the head portion 1 of the retrieval bag is automatically expanded under the action of the memory metal ring 4, and in this case, the specimen can be placed into the inner cavity 5 of the retrieval bag. Subsequently, the head portion 1 and the neck portion 2 of the retrieval bag are pulled out of the body surface from the trocar, the pneumoperitoneum is temporarily closed, and after the puncture cannula is removed, the trocar is immediately reinserted into the retrieval bag from the opening of the head portion 1 of the retrieval bag, and the pneumoperitoneum is reopened. After gas enters the inner cavity 5 containing the specimen through the puncture cannula, the air pressure inside the inner cavity 5 is higher than the air pressure of the external abdominal cavity, so that the inner cavity 5 is expanded to form a sufficiently large operating space, allowing an angle of the specimen to be accurately and quickly adjusted or the specimen to be efficiently and regularly segmented by a surgical instrument (e.g. an appendix with moderate to severe inflammation has a long strip shape and can be removed from a small incision after angle adjustment; alternatively, a gallbladder with an excessive burden on stone can be efficiently and regularly dissected to separate stones and bile, separating solid stones, soft tissues, and liquid bile, and ultimately achieving complete removal of the specimen tissue and avoiding irregular tearing of the specimen). Meanwhile, a part of gas entering the inner cavity 5 enters the outer cavity 6 through the air inlet hole 11 and is released into the abdominal cavity through the air outlet hole 10, so that the air pressure in the abdominal cavity is maintained in a state of being greater than the external air pressure, and a sufficient air cavity space is formed in the abdominal cavity. During this process, since the puncture hole in the abdominal wall is not completely sealed, the air in the abdominal cavity is also continuously released to the outside along with the puncture hole in the abdominal wall, so that a state that the air pressure of the inner cavity 5 of the retrieval bag is greater than the air pressure of the abdominal cavity and greater than the external air pressure is always maintained in a surgical process, and both the inner cavity 5 and the abdominal cavity have sufficient space for surgeons to perform surgical operations.

### Embodiment II

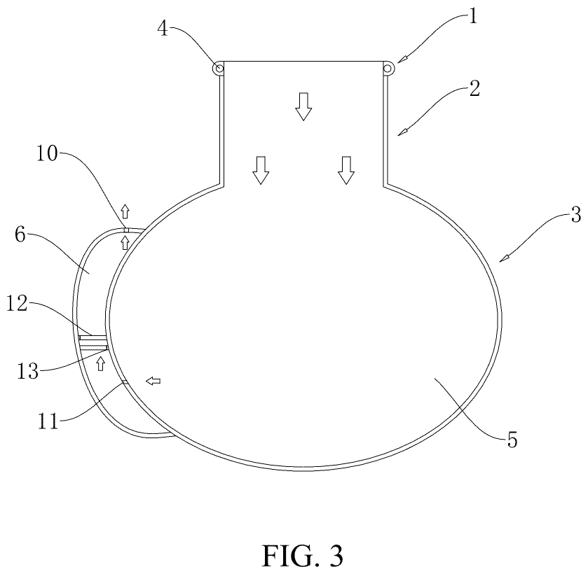

As shown in FIG. 3, the difference between the laparoscopic difficult specimen retrieval bag of this embodiment and that of Embodiment I lies in that an inner cavity 5 of a retrieval bag is an integral cavity and is not divided into an upper cavity 7 and a lower cavity 8. The outer cavity 6 is connected to an outer side surface of the inner cavity 5, and the outer cavity 6 is tubular as a whole. An air outlet hole 10 is formed in an upper part of the outer cavity 6, and an air inlet hole 11 in communication with the inner cavity 5 is formed in a middle part of the outer cavity 6. The air inlet hole 11 is connected to a position at a lower part in the inner cavity 5. At least two layers of isolation vanes 12 are disposed inside the outer cavity 6 at a position between the air inlet hole 11 and the air outlet hole 10, and a spacing is reserved between adjacent isolation vanes 12. The isolation vanes 12 partition the entire outer cavity 6, and a ventilation slit 13 is formed on each layer of the isolation vane 12. The ventilation slits 13 on adjacent isolation vanes 12 are disposed in a staggered manner.

### Embodiment III

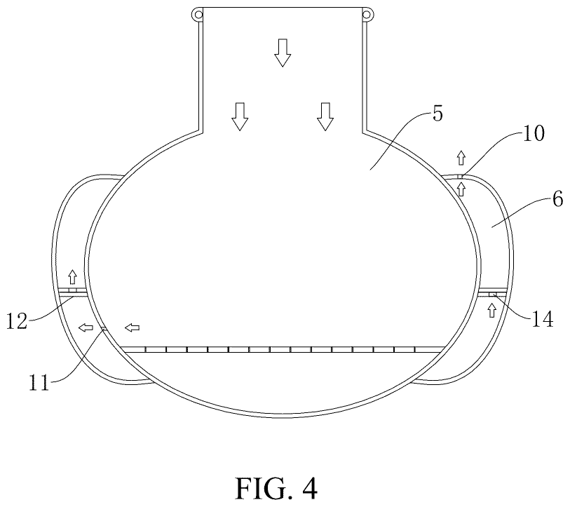

As shown in FIG. 4, the difference between the laparoscopic difficult specimen retrieval bag of this embodiment and that of Embodiment I lies in that the outer cavity 6 is a rotational body cavity surrounding the inner cavity 5. An air outlet hole 10 is formed in an upper part of the outer cavity 6, and an air inlet hole 11 in communication with the inner cavity 5 is formed in a middle part of the outer cavity 6. The air inlet hole 11 is connected to a position near a bottom of the upper cavity 7 and above the diaphragm 9. Both the air inlet hole 11 and the air outlet hole 10 are small holes and are respectively located on two sides of the inner cavity 5. Two layers of isolation vanes 12 are disposed at a position above the air inlet hole 11 inside the outer cavity 6. The isolation vanes 12 are annular and partition the entire outer cavity 6. The isolation vanes 12 are stacked together, and a ventilation hole 14 is cut and formed on each layer of the isolation vane 12. The ventilation hole 14 on the isolation vane 12 at a lower layer is located on the same side as the air outlet hole 10 relative to the inner cavity 5, and the ventilation hole 14 on the isolation vane 12 at an upper layer is located on the same side as the air inlet hole 11 relative to the inner cavity 5.

The above are preferred embodiments of this disclosure and are not intended to limit the scope of protection of this disclosure. Therefore, any equivalent changes made according to the structure, shape, and principle of this disclosure shall fall within the scope of protection of this disclosure.

What is claimed is:

1. A laparoscopic difficult specimen retrieval bag, comprising a head portion (1) with an opening on one side, a body portion (3), and a neck portion (2) connecting the head portion (1) and the body portion (3), wherein the body portion (3) comprises an inner cavity (5) and an outer cavity (6), the inner cavity (5) is in communication with the neck portion (2), the outer cavity (6) is provided with an air inlet hole (11) and an air outlet hole (10), the air inlet hole (11) is used for being in communication with the inner cavity (5), and the air outlet hole (10) is used for being in communication with the outside.

2. The laparoscopic difficult specimen retrieval bag according to claim 1, wherein a memory metal ring (4) for enabling the retrieval bag to automatically and elastically expand is disposed at the opening of the head portion (1).

3. The laparoscopic difficult specimen retrieval bag according to claim 1, wherein a diaphragm (9) is disposed in the inner cavity (5) to divide the inner cavity (5) into an upper cavity (7) and a lower cavity (8), and a pore or a hole is disposed on the diaphragm (9).

4. The laparoscopic difficult specimen retrieval bag according to claim 3, wherein the air inlet hole (11) is connected to a position in the inner cavity (5) near a bottom of the upper cavity (7) and above the diaphragm (9).

5. The laparoscopic difficult specimen retrieval bag according to claim 1, wherein the outer cavity (6) is tubular and is located on one side of the inner cavity (5), isolation vanes (12) are disposed inside the outer cavity (6) at a position between the air inlet hole (11) and the air outlet hole (10), and ventilation slits (13) are disposed on the isolation vanes (12).

6. The laparoscopic difficult specimen retrieval bag according to claim 5, wherein at least two layers of the isolation vanes (12) are disposed, and the ventilation slits (13) on adjacent isolation vanes (12) are disposed in a staggered manner.

7. The laparoscopic difficult specimen retrieval bag according to claim 1, wherein the air inlet hole (11) is connected to a position at a lower part in the inner cavity (5).

8. The laparoscopic difficult specimen retrieval bag according to claim 1, wherein the outer cavity (6) is a rotational body cavity surrounding the inner cavity (5), and the air inlet hole (11) and the air outlet hole (10) are respectively located on opposite sides relative to the inner cavity (5).

9. The laparoscopic difficult specimen retrieval bag according to claim 8, wherein two layers of isolation vanes (12) are disposed at a position above the air inlet hole (11) inside the outer cavity (6), and a ventilation hole (14) is disposed on each layer of the isolation vane (12).

10. The laparoscopic difficult specimen retrieval bag according to claim 9, wherein the two layers of the isolation vanes (12) are stacked together, the ventilation hole (14) on the isolation vane (12) at a lower layer is located on the same side as the air outlet hole (10) relative to the inner cavity (5), and the ventilation hole (14) on the isolation vane (12) at an upper layer is located on the same side as the air inlet hole (11) relative to the inner cavity (5).
